(19) United States
(12) Patent Application Publication
(10) Pub. No.: US 2019/0283007 A1
(43) Pub. Date: Sep. 19, 2019

(54) CATALYSTS FOR SOFT OXIDATION COUPLING OF METHANE TO ETHYLENE AND ETHANE

(71) Applicant: SABIC Global Technologies B.V., Bergen op Zoom (NL)

(72) Inventors: Gregory Biausque, Thuwal (SA); Jessica Scaranto, Thuwal (SA); Justin Johnson, Thuwal (SA); Khalid Albahily, Thuwal (SA)

(21) Appl. No.: 16/343,056

(22) PCT Filed: Oct. 26, 2017
(86) PCT No.: PCT/IB2017/056673

§ 371 Date: Apr. 18, 2019

Related U.S. Application Data
(60) Provisional application No. 62/414,872, filed on Oct. 31, 2016.

(51) Int. Cl. B01J 27/043; B01J 23/00; B01J 27/04; B01J 37/03; B01J 37/20; B01J 37/10; B01J 23/14; C07C 2/84; B01J 8/18

(52) U.S. Cl. CPC B01J 27/043; B01J 2208/00991; B01J 23/005; B01J 27/04; B01J 37/031; B01J 37/20; B01J 37/10; B01J 23/14; C07C 2/84; B01J 8/1827; B01J 2523/62; B01J 2523/72; B01J 2523/27; C07C 2527/04; C07C 2523/10; C07C 2523/14; C07C 2523/34; C07C 2527/043; C07C 2521/06; C07C 2523/02; B01J 23/002

(57) ABSTRACT

Disclosed is a catalyst and methods for the oxidative coupling of methane (OCM) reaction using elemental sulfur as a soft oxidant. The process can provide ethylene from methane with high conversion and selectivity.

### CROSS REFERENCE TO RELATED APPLICATIONS

This application claims the benefit of priority of U.S. Provisional Patent Application No. 62/414,872 filed Oct. 31, 2016, which is hereby incorporated by reference in its entirety.

### BACKGROUND OF THE INVENTION

### A. Field of the Invention

The invention generally concerns catalysts and methods to prepare and use the catalysts for the production of olefins from a soft oxidative coupling of methane reaction that uses methane and elemental sulfur as reactants. The catalysts can be supported or bulk catalysts, and can include a metal, a metal oxide, a lanthanide sulfide, an oxysulfide, or any combination thereof.

### B. Description of Related Art

Ethylene is one of the world's largest commodity chemicals and the chemical industry's fundamental building block. For example, ethylene derivatives are typically found in food packaging, eyeglasses, cars, medical devices, lubricants, engine coolants, and liquid crystal displays. For industrial scale applications, ethylene is currently produced by heating natural gas condensates and petroleum distillates, which include ethane and higher hydrocarbons. The produced ethylene is separated from the product mixture using gas separation processes. FIG. 1 provides an example of products generated from ethylene.

Ethylene can also be produced by oxidative coupling of methane (OCM) using sulfur as a soft oxidant. By way of example, U.S. Pat. No. 9,403,737 to Marks et al. describes the use of sulfur vapor with palladium sulfide, palladium subsulfides, molybdenum sulfide, titanium sulfide, ruthenium sulfide, or tantalum sulfide to catalyze the OCM reaction. The most active catalyst, PdS, was reported to yield a 16% conversion and 20% ethylene selectivity. In another example, Peter et al., in J. Am. Chem. Soc., 2015, 137(48), 15234-15240, used Pd/Fe₃O₄ or oxides of Mg, Zr, Sm, W, Ti, Fe, Cr, and La catalysts. The Mg, Zr, Sm, W, and La catalysts suffered in that they coked during the reaction. Furthermore, all of these catalysts suffered from low methane conversion. In yet another example, International Application Publication No. WO/2016/134305 to Marks et al. describes using metal chalcogenide catalysts for oxidative coupling of methane using sulfur as an oxidant. The most active catalyst, Sm₂O₃, was reported to yield a 11% conversion and 35% ethylene selectivity.

While there have been a few attempts to utilize sulfur as a soft oxidant for the OCM reaction, none have reported ethylene conversions and selectivities that would be useful for industrial applications.

### SUMMARY OF THE INVENTION

A solution to the problems associated with catalysts used in the oxidative coupling of methane (OCM) using sulfur as a soft oxidant have been discovered. The solution resides in contacting a reaction mixture including methane (CH₄) and elemental sulfur gas with a metal catalyst to produce an olefin, such as ethylene (C₂H₄) as shown in the reaction equation (1):

2CH₄+S₂→C₂H₄2H₂S (1)

The catalyst of the current invention can be a metal, a mixed metal sulfide, a metal oxysulfide, mixed metal oxysulfide, mixed metal oxide or any combination thereof. The catalyst can have a spinel-type structure (e.g., A²⁺B₂³⁺O_{4-y}²⁻S_{y}²⁻ or B₂O_{3-y}²⁻S_{y}²⁻), a halite-type structure (e.g., A₁₋ₓBₓO_{1-y}S_{y}), a rutile-type structure (e.g., ABO_{2-y}S_{y}), a perovskite-type structure (e.g., ABO_{3-y}²⁻S_{y}²⁻ or A²⁺(B′ₓB₍₁₋ₓ₎)⁴⁺O_{3-y}²⁻S_{y}²). The catalysts of the current invention have better conversion and selectivity than the conventional catalysts, (e.g., Pd/Fe₃O₄, palladium sulfide, palladium subsulfides, molybdenum sulfide, titanium sulfide, ruthenium sulfide, tantalum sulfide oxides of Mg, Zr, Sm, W, Ti, Fe, Cr, and La catalysts, or metal chalcogenide (e.g., sulfur (S), selenium (Se) or tellurium (Te)). This increased conversion and selectivity can allow for industrial scale production of olefins (e.g., ethylene) from methane and elemental sulfur.

In a particular aspect of the invention, there is disclosed a method of producing olefins from methane and elemental sulfur. The method can include: (a) obtaining a reaction mixture comprising methane and elemental sulfur gas; and (b) contacting the reaction mixture with a catalyst of the present invention under reaction conditions sufficient to produce a product stream comprising an olefin. In one aspect, the olefin comprises C₂+ hydrocarbons, preferably ethylene. In another aspect, the product stream further includes hydrogen sulfide. In certain aspects, the reaction mixture includes a methane to elemental sulfur molar ratio of 1:2 to 20:1, preferably about 15:2. The conditions sufficient to produce a product stream in step (b) of the method can include a reaction temperature of at least 450° C. or 600° C. to 1100° C., preferably 750° C. to 950° C. and a reaction pressure of 0.05 to 10.0 MPa or 0.1 to 10.0 MPa, preferably 0.5 to 2.5 MPa. In some aspects, the conditions of the method include a gas hourly space velocity (GHSV) of 500 to 100,000 h⁻¹ or 1,000 to 50,000 h⁻¹, preferably 3500 to 10,000 h⁻¹. The catalyst of the present invention can include a metal, a mixed metal oxide, a mixed metal sulfide, a metal oxysulfide, mixed metal oxysulfide, or any combination thereof. The metal, the mixed metal oxide, the metal oxysulfide, mixed metal oxysulfide, or the mixed metal sulfide can include an alkaline earth metal (e.g., magnesium (Mg), calcium (Ca), strontium (Sr), barium (Ba), or any combination thereof), a transition metal (e.g., yttrium (Y), zirconium (Zr), vanadium (V), tantalum (Ta), tungsten (W), manganese (Mn), rhenium (Rh), iron (Fe), cobalt (Co), iridium (Ir), nickel (Ni), copper (Cu), zinc (Zn), or any combination thereof), a post-transition metal (e.g. aluminum (Al), gallium (Ga), indium (In), silicon (Si), germanium (Ge), tin (Sn), antimony (Sb), bismuth (Bi), or any combination thereof), a lanthanide (e.g., lanthanum (La), cerium (Ce), praseodymium (Pr), neodymium (Nd), samarium (Sm), europium (Eu), gadolinium (Gd), or any combination thereof), or any combination thereof. The catalyst of the present invention can have a spinel-, halite-, rutile-, or fluorite-structure having one or more metals A and one or metals B or B′ where metals A, B, and/or B′ with each being individually chosen from alkaline earth metal, a transition metal, a post-transition metal, or a lanthanide. In some embodiments the catalyst has a perovskite-type structure where A, B can each independently be one or more of an alkaline earth metal, a transition metal, a post-transition metal or a lanthanide metal and B′ is an alkaline earth metal, a transition metal, a post-transition metal or a lanthanide metal. In a preferred instance, metal A has a total oxidation state of +2, while metal B, B′, or B₂ has an oxidation state of +3 to +6 and can change oxidation states to accommodate oxygen and/or sulfur through vacancies. Still further, catalysts of the present invention can be an ordered mixture catalyst including a superstructure obtained by intercalation or substitution of elements. In some instances, the catalyst can include a bulk metal catalyst or a supported catalyst. In other instances, the catalyst is a supported catalyst and the support can include alumina, silica, titania, zirconia, magnesia, lime, silicon carbide, or combinations thereof. The support can be macroporous, mesoporous, microporous, or any combination thereof.

In another particular aspect of the invention, there is disclosed a system for producing olefins from alkanes and elemental sulfur. The system can include an inlet for a feed including a gaseous alkane(s) and elemental sulfur gas or a first inlet for a feed comprising a gaseous alkane(s) and a second inlet for a feed including elemental sulfur gas, and a reactor including a reaction zone that is configured to be in fluid communication with the inlet or inlets. The reaction zone can include gaseous alkane(s), elemental sulfur gas, and a catalyst of the present invention capable of catalyzing the reaction between the alkane(s) and the sulfur gas to produce a product stream comprising a gaseous olefin(s); and an outlet configured to be in fluid communication with the reaction zone to remove the product stream from the reactor.

In a particular aspect of the invention, 20 embodiments are described. Embodiment 1 describes a method of producing an olefin from methane and elemental sulfur, the method can include: (a) obtaining a reaction mixture comprising methane and elemental sulfur gas; and (b) contacting the reaction mixture with a catalyst under reaction conditions sufficient to produce a product stream comprising an olefin, wherein the catalyst is a metal, a mixed metal oxide, mixed metal sulfide, a metal oxysulfide, mixed metal oxysulfide, or any combination thereof. Embodiment 2 is the method of embodiment 1, wherein the olefin comprises C₂+ hydrocarbons, preferably ethylene. Embodiment 3 is the method of any one of embodiments 1 to 2, wherein the product stream further comprises hydrogen sulfide. Embodiment 4 is the method of any one of embodiments 1 to 3, wherein the reaction mixture comprises a methane to elemental sulfur molar ratio of 1:2 to 20:1, preferably 5:1 to 10:1, or more preferably 7.5:1. Embodiment 5 is the method of any one of embodiments 1 to 4, wherein the conditions sufficient to produce a product stream in step (b) comprise a reaction temperature of at least 450° C. or 600° C. to 1100° C., preferably 750° C. to 950° C. Embodiment 6 is the method of any one of embodiments 1 to 5, wherein the conditions sufficient to produce a product stream comprise a reaction pressure of 0.05 to 10.0 MPa or 0.1 to 10.0 MPa, preferably 0.5 to 2.5 MPa, a gas hourly space velocity (GHSV) of 500 to 100,000 h⁻¹ r 1,000 to 50,000 h⁻¹, preferably 3500 to 10,000 h⁻¹ or both. Embodiment 7 is the method of embodiment 1, wherein the metal, the mixed metal oxide, the mixed metal sulfide, the metal oxysulfide, mixed metal oxysulfide, or the metal sulfide comprises: an alkaline earth metal, preferably magnesium (Mg), calcium (Ca), strontium (Sr), barium (Ba), or any combination thereof; a transition metal, preferably yttrium (Y), zirconium (Zr), vanadium (V), tantalum (Ta), tungsten (W), manganese (Mn), rhenium (Rh), iron (Fe), cobalt (Co), iridium (Ir), nickel (Ni), copper (Cu), zinc (Zn), or any combination thereof; a post-transition metal, preferably aluminum (Al), gallium (Ga), indium (In), silicon (Si), germanium (Ge), tin (Sn), antimony (Sb), bismuth (Bi), or any combination thereof; a lanthanide, preferably, lanthanum (La), cerium (Ce), praseodymium (Pr), neodymium (Nd), samarium (Sm), europium (Eu), gadolinium (Gd), or any combination thereof; or any combination thereof. Embodiment 8 is the method of any one of embodiments 1 to 7, wherein the catalyst does not include platinum sulfide, palladium sulfide molybdenum sulfide, titanium sulfide, ruthenium sulfide, tantalum sulfide, or combinations thereof. Embodiment 9 is the method of any one of embodiments 1 to 8, wherein the catalyst does not include a metal oxide, preferably MgO, ZrO₂, TiO₂, CeO₂, Sm₂O₃, ZnO, W₂O₃, Cr₂O₃, La₂O₃ and Fe₃O₄. Embodiment 10 is the method of any one of embodiments 1 to 9, wherein the catalyst comprises a spinel-, a halite-, a rutile-, or a perovskite-type crystal structure, or any combination thereof. Embodiment 11 is the method of embodiment 10, wherein the catalyst is an ordered mixture of one or more of the spinel-, halite-, rutile-, fluorite- or perovskite-type crystal structure, preferably a superstructure. Embodiment 12 is the method of any one of embodiments 1 to 11, wherein the catalyst has a spinel-type structure with a general formula of A²⁺B₂³⁺O_{4-y}²⁻S_{y}²⁻ where 0≤y≤4, or B₂O_{3-y}²⁻S_{y}²⁻ where 0≤y≤3, or A²⁺B′ₓ⁺³B₍₂₋ₓ₎³⁺O_{4-y}²⁻S_{y}²⁻ where 0≤x≤2 and 0≤y≤4 and A, B, and B′ are each independently an alkaline earth metal, a transition metal, a post transition metal or a lanthanide metal, preferably ZnMn₂O₄₋S_{y}, CuFe₂O_{4-y}S_{y}, SrIn₂O_{4-y}S_{y}, ZnGa₂O_{4-y}S_{y}, CoBiₓFe₍₂₋ₓ₎O_{4-y}S_{y}, MgGe₂O_{4-y}S_{y}, where 0≤x≤2 and 0≤y≤4 or Gd₂O_{3-y}S_{y} where 0≤y≤3. Embodiment 13 is the method any one of embodiments 10 to 11, wherein the catalyst has a halite-type structure with a general formula A₁₋ₓBₓO_{1-y}S_{y}, where 0≤x≤1 and 0≤y≤1, and where A and B are each independently an alkaline earth metal, a transition metal, a post transition metal, or a lanthanide metal, preferably MnO_{1-y}S_{y}, Co_{0.2}Ni_{0.80}O_{1-y}S_{y}, ZnO_{1-y}S_{y}, or EuO_{1-y}S_{y}. Embodiment 14 is the method any one of embodiments 10 to 11, wherein the catalyst comprises a rutile-type structure with a general formula of A₁₋ₓBₓO_{2-y}S_{y}, where 0≤x≤1 and 0≤y≤2, and A and B are each independently an alkaline earth metal, a transition metal, a post transition metal, or a lanthanide metal, preferably FeO_{2-y}S_{y}, GeO_{2-y}S_{y}, or GdO_{2-y}S_{y}, where 0≤y≤2. Embodiment 15 is the method of any one of embodiments 10 to 11, wherein the catalyst comprises a perovskite-type structure with a general formula ABO_{3-y}²⁻S_{y}²⁻ where 0≤y≤3, and A and B are each independently an alkaline earth metal, a transition metal, a post transition metal, or a lanthanide metal, preferably CaGeO_{3-y}S_{y}, LaNbO_{3-y}S_{y}, PrNiO_{3-y}S_{y}, or NdGaO_{3-y}S_{y}, where 0≤y≤3, or a perovskite-type structure with a general formula A²⁺(B′ₓB₍₁₋ₓ₎)⁴⁺O_{3-y}²⁻S_{y}², wherein A, B can each independently be one or more of an alkaline earth metal, a transition metal, a post-transition metal or a lanthanide metal, 0.1≤x≤0.9, 0≤y≤3, and B′ is an alkaline earth metal, a transition metal, a post-transition metal or a lanthanide metal. Embodiment 16 is the method of any one of embodiments 10 to 11, wherein the catalyst comprises a fluorite-type structure with a general formula AO₂₋ₓSₓ, ABO_{3.5-y}S_{y}, or A₂O_{3-z}S_{z}, where 0≤x≤2, 0≤y≤3.5, 0≤z≤3, and A and B are each independently an alkaline earth metal, a transition metal, a post transition metal, or a lanthanide metal, preferably Bi₂O_{3-z}S_{z} where 0≤z≤3. Embodiment 17 is the method of any one of embodiments 9 to 16, wherein A and B are each individually an alkaline earth metal, a transition metal, a post-transition metal, or a lanthanide; wherein A is a 2+ charged cation, preferably calcium (Ca), strontium (Sr), europium (Eu), indium (In), gallium (Ga), zinc (Zn), nickel (Ni), cobalt (Co), or copper (Cu), and B, B₂, B′, or a combination thereof are a 3+ to 6+ charged cation that can change oxidation state to accommodate oxygen and/or sulfur, preferably manganese (Mn), iron (Fe), germanium (Ge), cerium (Ce), or bismuth (Bi). Embodiment 18 is the method of any one of embodiments 1 to 17, wherein the catalyst is a bulk metal catalyst or a supported catalyst. Embodiment 19 is the method of embodiment 18, wherein the catalyst is a supported catalyst and the support comprises alumina, silica, titania, zirconia, magnesia, lime, silicon carbide, or combinations thereof, and, optionally, the support is macroporous, mesoporous, microporous, or any combination thereof. Embodiment 20 is a system for producing olefins from alkanes and elemental sulfur, the system can include: an inlet for a feed comprising a gaseous alkane(s) and elemental sulfur gas or a first inlet for a feed comprising a gaseous alkane(s) and a second inlet for a feed comprising elemental sulfur gas; and a reactor comprising a reaction zone that is configured to be in fluid communication with the inlet or inlets, wherein the reaction zone comprises gaseous alkane(s), elemental sulfur gas, and a catalyst capable of catalyzing the reaction between the alkane(s) and the sulfur gas to produce a product stream comprising a gaseous olefin(s), wherein the catalyst is a metal, a mixed metal oxide, mixed metal sulfide, a metal oxysulfide, a mixed metal oxysulfide, or any combination thereof; and an outlet configured to be in fluid communication with the reaction zone to remove the product stream from the reactor.

The following includes definitions of various terms and phrases used throughout this specification.

The term “catalyst” means a substance, which alters the rate of a chemical reaction. “Catalytic” means having the properties of a catalyst.

The phrase “mixed metal oxide” refers to a solid solution (one crystal structure) or composite (at least two crystal structures) composed of two of more elements from an alkaline metal, alkaline earth metal, transition metal, metalloids, lanthanides, or actinides of the Periodic Table in a non-zero oxidation state denoted as metallic cations bonded with an equimolar amount of oxo-anions O²⁻ in order to keep the mixed metal oxide overall neutral in terms of charge. “Mixed metal oxide” does not include individual metal oxides that are merely mixed together (i.e., that are mixed together as a solid-solid mixture but not present in the same framework of a crystal lattice structure).

The phrase “mixed metal sulfide” refers refers to a solid solution (one crystal structure) or composite (at least two crystal structures) composed of two of more elements from an alkaline metal, alkaline earth metal, transition metal, metalloids, lanthanides, or actinides of the Periodic Table in a non-zero oxidation state denoted as metallic cations bonded with an equimolar amount of sulfide S²⁻ in order to keep the mixed metal sulfide overall neutral in terms of charge. “Mixed metal sulfide” does not include individual metal sulfides that are merely mixed together (i.e., that are mixed together as a solid-solid mixture, but do not have two metals present in the same framework of the crystal lattice structure).

The term “conversion” means the mole fraction (i.e., percent) of a reactant converted to a product or products.

The term “selectivity” refers to the percent of converted reactant that went to a specified product. In a non-limiting example, C₂+ hydrocarbon selectivity is the % of methane that formed ethane, ethylene, and higher hydrocarbons.

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having,” in the claims or the specification may mean “one,” but it is also consistent with the meaning of “one or more,” “at least one,” and “one or more than one.”

The terms “wt. %”, “vol. %”, or mol % refers to a weight, volume, or molar percentage of a component, respectively, based on the total weight, the total volume, or the total moles of material that includes the component. In a non-limiting example, 10 grams of component in 100 grams of the material that includes the component is 10 wt. % of component.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The methods of the present invention can “comprise,” “consist essentially of,” or “consist of” particular ingredients, components, compositions, etc. disclosed throughout the specification. With respect to the transitional phase “consisting essentially of,” in one non-limiting aspect, a basic and novel characteristic of the methods of the present invention is their capability to production of olefins (e.g., ethylene) from alkanes (e.g., methane) and elemental sulfur with selectivity and conversion parameters that can allow for industrial scale production of olefins.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description.

### BRIEF DESCRIPTION OF THE DRAWINGS

FIG. 1 depicts an illustration of various chemicals and products that can be produced from ethylene.

FIG. 2 is a schematic of a system of the present invention using the catalyst of the present invention in an oxidative coupling of methane reaction using elemental sulfur as a soft oxidant.

### DETAILED DESCRIPTION OF THE INVENTION

Currently available processes to produce light olefins (e.g., ethylene) by OCM using sulfur as a soft oxidant are often thwarted by poor performance (i.e., low conversion and selectivities). Low methane conversion and light olefin selectivity (e.g., ethylene) makes commercial/industrial scale operation impractical or unfeasible. A discovery has been made that results in increased methane conversion and increased light olefin selectivity for the OCM reaction where elemental sulfur is used as an oxidant as compared to conventional catalysts (e.g., Pd/Fe₃O₄, palladium sulfide, palladium subsulfides, molybdenum sulfide, titanium sulfide, ruthenium sulfide, tantalum sulfide oxides of Mg, Zr, Sm, W, Ti, Fe, Cr, and La catalysts, and metal chalcogenides). The discovery is based, in part, on the identification of particular reaction conditions and/or particular catalysts.

These and other non-limiting aspects of the present invention are discussed in further detail in the following sections.

### A. Catalysts

The catalysts of the present invention can include a catalytic metal material and an optional support material.

### 1. Catalytic Material

The catalytic metal can be a metal, a mixed metal oxide, a metal oxysulfide, mixed metal oxysulfide, or a mixed metal sulfide containing an alkaline earth metal, a transition metal, a post-transition metal, a lanthanide, or any combination thereof from Columns 2 to 13 of the Periodic Table. Preferable transition metals include yttrium (Y), zirconium (Zr), vanadium (V), niobium (Nb), tantalum (Ta), tungsten (W), manganese (Mn), rhenium (Rh), iron (Fe), cobalt (Co), iridium (Ir), nickel (Ni), copper (Cu), zinc (Zn) or any combination thereof. Preferable lanthanides include lanthanum (La), cerium (Ce), praseodymium (Pr), neodymium (Nd), samarium (Sm), europium (Eu), gadolinium (Gd), or combinations thereof. Preferable post-transition metals include aluminum (Al), gallium (Ga), indium (In), silicon (Si), germanium (Ge), tin (Sn), antimony (Sb), bismuth (Bi), manganese (Mn) or any combination thereof. Preferable alkaline earth metals include magnesium (Mg), calcium (Ca), strontium (Sr), barium (Ba) or any combination thereof.

The catalytic material can have various crystal structures such as spinel-, halite-, rutile-, or perovskite-type crystal structures, which are described in more detail below. Still further, the catalytic material can include a superstructure containing any of spinel-type, halite-type, rutile-type, or perovskite-type structures obtained by intercalation or substitution of elements in the crystal structure.

In certain aspects, the catalytic material can have a spinel-type crystal structure with the general formula of A²⁺B³⁺O_{4-y}²⁻S_{y}²⁻, where 0≤y≤4, or B₂O_{3-y}S_{y} where 0≤y≤3, or A²⁺B′ₓ⁺³B₍₂₋ₓ₎³⁺O_{4-y}²⁻S_{y}²⁻ where 0≤x≤2 and 0≤y≤4. In some embodiments y is 0, 1, 2, 3, 4, or any number there between. Spinel-type structures can have a cubic (isometric) crystal structure. Non-limiting examples of spinel-type catalyst of the present invention include ZnMn₂O_{4-y}S_{y}, CuFe₂O_{4-y}S_{y}, SrIn₂O_{4-y}S_{y}, ZnGa₂O_{4-y}S_{y}, MgGe₂O_{4-y}S_{y}, where 0≤y≤4, or Gd₂O_{3-y}S_{y} where 0≤y≤3 or CoBiₓFe₍₂₋ₓ₎O_{4-y}S_{y} where 0≤x≤2 and 0≤y≤4. In some embodiments y is 0, 1, 2, 3, 4, or any number there between and/or x is 0, 1, 2 or any number there between.

In other certain aspects, the catalytic material can have a halite-type structure with the general formula of A₁₋ₓBₓO_{1-y}S_{y}, where 0≤x≤1 and 0≤y≤1. In some embodiments y is 0, 1, or any number there between and/or x is 0, 1, or any number there between. A halite or “rock salt” structure can be similar to the space group of NaCl (rock salt). The unit cell of the crystal structure can be in the shape of a cube (e.g., cubic or isometric crystal). Non-limiting examples of catalysts of the present invention having a halite-type structure include MnO_{1-y}S_{y}, Co_{0.2}Ni_{0.80}O_{1-y}S_{y}, ZnO_{1-y}S_{y}, or EuO_{1-y}S_{y}, where 0≤y≤1.

In other instances, the catalytic material can have a rutile-type structure with the general formula of A₁₋ₓBₓO_{2-y}S_{y}, where 0≤x≤1 and 0≤y≤2. In some embodiments, y is 0.001, 1, 2, or any number there between and/or x is 0.001, 1, or any number there between. A rutile-type structure can have a body-centered tetragonal unit cell. Non-limiting examples of catalytic material of the present invention having a rutile-type structure include FeO_{2-y}S_{y}, GeO_{2-y}S_{y}, or GdO_{2-y}S_{y}, where 0≤y≤2.

In another instance, the catalytic material can have fluorite-type structure with the general formula AO₍₂₋ₓ₎Sₓ, ABO_{(3.5-y)}S_{y}, or A₂O_{(3-z)}S_{z}, where 0≤x≤2, 0≤y≤3.5, 0≤z≤3. In some embodiments y is 0, 0.5, 1, 1.5, 2, 2.5, 3, 3.5, or any number there between, x is 0, 1, 2 or any number there between, and z is 0, 1, 2, 3, or any number there between. A fluorite-type structure can have face-center cubit unit cell. A non-limiting example of catalytic material of the present invention having a fluorite-type structure includes Bi₂O_{3-z}S_{z} where 0≤z≤3.

In still other instances, the catalytic material can have a perovskite-type structure. A perovskite-type structure can have a cubic crystal (perovskite) structure having a general formula of ABO₃, which may be structured in layers and many structural formulas. In one instance, a perovskite-type structure can have a general formula of A²⁺B⁴⁺O_{3-y}²⁻S_{y}²⁻, where 0≤y≤3 or A³⁺B³⁺O_{3-y}²⁻S_{y}²⁻, where 0≤y≤3. In some embodiments y is 0.001, 1, 2, 3, or any number there between. Non-limiting examples of catalyst of the present invention having a perovskite-type structure can include Ca²⁺Ge⁴⁺O_{3-y}S_{y}, La²⁺Nb⁴⁺O_{3-y}S_{y}, Pr³⁺Ni³⁺O_{3-y}S_{y}, or Nd³⁺Ga³⁺O_{3-y}S_{y}, where 0≤y≤3. In one preferred instance, the catalytic material is PrNiO_{3-y}S_{y} where 0≤y≤3. In another instance, the perovskite-type structure can have an empirical chemical formula of A²⁺(B′ₓB₍₁₋ₓ₎)⁴⁺O_{3-y}²⁻S_{y}², wherein A, B can each independently be one or more of an alkaline earth metal, a transition metal, a post-transition metal or a lanthanide metal, 0.1≤x≤0.9, and 0≤y≤3, and B′ is alkali metal, an alkaline earth metal, a transition metal, a post-transition metal or a lanthanide metal. B′, in some embodiments, can be considered a dopant. In some embodiments, x is 0.1, 0.2, 0.3, 0.4, 0.5, 0.6, 0.7, 0.8, 0.9, or any number there between and y is 0.001, 1, 2, 3, or any number there between. A non-limiting example of these perovskite-type catalysts includes Ca²⁺(NaₓNb₍₁₋ₓ₎)O_{3-y}²⁻S_{y}². In some embodiments, the net charge of the (B′ₓB₁₋ₓ) complex is +3 or +4, however, the net charge may vary with oxygen content and sulfur content.

In certain aspects, the other catalysts of the present invention can also be promoted with a dopant. Non-limiting examples of dopants can include aluminum (Al), chlorine (Cl), copper (Cu), iron (Fe), magnesium (Mg), niobium (Nb), nickel (Ni), palladium (Pd), platinum (Pt), antimony (Sb), tantalum (Ta), zinc (Zn), zirconium (Zr), or combinations thereof. A dopant is a species, which is intentionally introduced into an intrinsic material in order to produce some effect. Unintentional impurities which exist in concentrations below approximately 0.01 mole percent are not generally considered dopants.

Without being limited to theory, any of the spinel-, halite-, rutile-, fluorite-, or perovskite-type structure general formulas described throughout the specification can include metals A, A′, B, and/or B′ with each being individually chosen from alkaline earth metal, a transition metal, a post-transition metal, or a lanthanide. In some instances, A has a total charge of 2+ (e.g., calcium, magnesium, strontium, europium, indium, gallium, zinc, nickel, cobalt and copper), while B, B′, or B₂ has a total charge of 3+ to 6+ (e.g., manganese, praseodymium, iron, germanium, cerium, and bismuth) that can change oxidation states easily and accommodate oxygen and/or sulfur through vacancies. In a preferred instance, metal A has a total oxidation state of +2, while metal B, B′, or B₂ has an oxidation state of +4 to +6 and can change oxidation states to accommodate oxygen and/or sulfur through vacancies.

The amount of catalytic material in the catalyst depends, inter alia, on the desired catalytic activity of the catalyst. In some aspects, the amount of catalytic material present in the catalyst ranges from 1 to 100 parts by weight of catalytic material per 100 parts by total weight of catalyst or from 10 to 50 parts by weight of catalytic material per 100 parts by weight of total catalyst. In a non-limiting aspect, the amount of catalytic material present ranges from 5 to 20 parts by weight of catalytic material per 100 parts by weight of catalyst and all parts by weight there between including 6, 7, 8, 9, 10, 11, 12, 13, 14, 15, 16, 17, 18, and 19 parts by weight (wt. %). Without being limited by theory, the amount of catalytic material in the catalyst can be controlled by the amount of support material. A non-limiting commercial source of the metals for use in the current invention includes Sigma-Aldrich®, (U.S.A.), Alfa Aesar (U.S.A.), and Fischer Scientific (U.S.A.).

The catalytic material can be produced and then sized to have micronized or nanosized particles or structures, or combinations thereof, using known sizing methods (e.g., granulation or powderification).

### 2. Support Material

In some aspects, the catalysts of the current invention can be supported. The support material or a carrier can be porous and/or have a high surface area. In some aspects, the support is active (i.e., has catalytic activity). In other aspects, the support is inactive (i.e., non-catalytic). In some aspects, the support can include an inorganic oxide, silicon dioxide (SiO₂), alpha, beta or theta alumina (Al₂O₃), activated Al₂O₃, titanium dioxide (TiO₂), magnesium oxide (MgO), calcium oxide (CaO), strontium oxide (SrO), zirconium oxide (ZrO₂), zinc oxide (ZnO), lithium aluminum oxide (LiAlO₂), magnesium aluminum oxide (MgAlO₄), manganese oxides (MnO, MnO₂, Mn₂O₄), lanthanum oxide (La₂O₃), activated carbon, silica gel, zeolites, activated clays, lime, carbides, silicon carbide (SiC), diatomaceous earth, magnesia, aluminosilicate, calcium aluminate, a carbonate (e.g., MgCO₃, CaCO₃, SrCO₃, BaCO₃, Y₂(CO₃)₃, or La₂(CO₃)₃), or combinations thereof. The support can be macroporous, mesoporous, microporous, or a combination thereof. In some instances, the support material can further contain, or can be further doped with, an alkali metal salt or alkaline earth metal (i.e., Columns 1 or 2 of the Periodic Table) or salt thereof. Non-limiting examples of metals include sodium (Na), lithium (Li), potassium (K), cesium (Cs), magnesium (Mg), calcium (Ca), barium (Ba), or combinations thereof.

All of the materials used to make the supported catalysts of the present invention can be purchased or made by processes known to those of ordinary skill in the art (e.g., precipitation/co-precipitation, sol-gel, templates/surface derivatized metal oxides synthesis, solid-state synthesis, of mixed metal oxides, microemulsion technique, solvothermal, sonochemical, combustion synthesis, etc.).

### 3. Method to Make the Catalyst

The catalysts of the current invention can be prepared by various methods. In one aspect, the method of preparation can include co-precipitation of catalytic material precursor or precursors (e.g., nitrate, chloride, acetate, carbonate, and sulfate) in a protic solvent using a precipitating agent such as sodium hydroxide, lithium hydroxide, ammonium hydroxide, ammonium carbonate, ammonium bicarbonate, etc. The resulting solid can be collected by filtration, dried, and calcined at a suitable temperature. In another aspect, the method of preparation can include solid-state chemistry where the catalytic material oxides are ground or milled together at high energy. The resulting solid can be dried and calcined at a suitable temperature. In yet another aspect, the method of preparation can include sol-gel chemistry where the catalytic material precursor or precursors are dissolved in a protic solvent to react with an organic molecule (e.g., a carboxylic acid or amine) to form a catalytic material complex. Further application of energy (e.g., thermal) to the catalytic material complex can result in a polymerization-type coordination of the complex and can evaporate the solvent. The resultant gel can be dried and calcined at a suitable temperature. In still another aspect, the method of preparation can include impregnation of the catalytic material precursor or precursors in solution to form a supported catalyst. In other aspects, a combination of catalysts can be intercalated or heated together to create mixtures of catalyst structures. The mixture can be an ordered mixture of the above-described structures that are intercalated.

Supported catalysts may be prepared using generally known catalyst preparation techniques. In some instances, impregnation can be achieved by dry (without solvent) or wet (with solvent) techniques. The resultant wet or dry solid can, if necessary, be dried, and then calcined at a suitable temperature. In any of the above methods, the materials may be mixed together using suitable mixing equipment. Examples of suitable mixing equipment include tumblers, stationary shells or troughs, Muller mixers (for example, batch type or continuous type), impact mixers, and any other generally known mixers, or generally known devices that can suitably provide the catalysts of the current invention. For solution chemistries, a mechanical stirrer or magnetic stir bar or can be used.

In a first non-limiting aspect, a suitable condition for dying catalysts prepared by solution, gel, or solid methods can include a temperature from 50° C. to 300° C. for 1 to 24 hours in air or under vacuum. Preferably, suitable conditions for drying include a temperature from 120° C. to 220° C. for 2 to 4 hours. In a second non-limited aspect, a suitable temperature for calcination of the isolated and dried catalyst can include subjecting the amorphous or crystalline material to a temperature of 350° C. to 1100° C. under an oxygen source or an inert atmosphere, preferably 700° C. to 1100° C. for 3 hours in the presence of an oxygen source (e.g., air).

### B. Reactants

The reactant mixture used to make olefins in the context of the present invention can be a gaseous mixture that includes, but is not limited to, a hydrocarbon or mixtures of hydrocarbons and sulfur gas (S(g)). Alternatively, the hydrocarbon or mixtures of hydrocarbons and S(g) feeds can be introduced separately and mixed in a reactor. The hydrocarbon or mixtures of hydrocarbons can include natural gas, liquefied petroleum gas containing of C₂ to C₅ hydrocarbons such as ethylene, ethane, propane, propylene, butane, butylene, isobutene, pentane and pentene, C₆+ heavy hydrocarbons (e.g., C₆ to C₂₄ hydrocarbons such as diesel fuel, jet fuel, gasoline, tars, kerosene, etc.), oxygenated hydrocarbons, and/or biodiesel, alcohols, or dimethyl ether, or combinations thereof. In some aspects, the hydrocarbon is a mixture of hydrocarbons that is predominately methane (e.g., natural gas). In even more preferred instances, the hydrocarbon consists of methane.

Sulfur gas (S(g)) in the context of the present invention can be referred to as elemental sulfur and can include, but is not limited to, all allotropes of sulfur (e.g., Sₙ where n=1 to ∞). Non-limiting examples of sulfur allotropes include S, S₂, S₄, S₆, and S₈, with the most common allotrope being S₈. Sulfur gas can be obtained by heating solid or liquid sulfur to a boiling point of about 445° C. Alternatively, gaseous sulfur can be generated by heating elemental sulfur in a sealed container and the gaseous sulfur can then be added to the reactor or mixed with the reactant gas feed. Solid sulfur can contain either (a) sulfur rings, which may have 6, 8, 10 or 12 sulfur atoms, with the most common form being S₈, or (b) chains of sulfur atoms, referred to as catena sulfur having the formula S. Liquid sulfur is typically made up of Ss molecules and other cyclic molecules containing a range of six to twenty atoms. Solid sulfur is generally produced by extraction from the earth using the Frasch process, or the Claus process. The Frasch process extracts sulfur from underground deposits. The Claus process produces sulfur through the oxidation of hydrogen sulfide (H₂S). Hydrogen sulfide can be obtained from waste or recycle stream (for example, from a plant on the same site, or as a product from hydrodesulfurization of petroleum products) or recovery the hydrogen sulfide from a gas stream (for example, separation for a gas stream produced during production of petroleum oil, natural gas, or both). A benefit of using sulfur as a starting material is that it is abundant and relatively inexpensive to obtain as compared to, for example, oxygen gas.

The reactant mixture may further contain other gases, preferably other gases that do not negatively affect the reaction (e.g., reduced conversion and/or reduced selectivity). Examples of such other gases include nitrogen or argon. In some aspects of the invention, the reactant gas stream can be substantially devoid of other reactant gas such as oxygen gas, carbon dioxide gas, hydrogen gas, water or any combination thereof. Preferably, the reactant mixture is highly pure and substantially devoid of water. In some embodiments, the gases can be dried prior to use (e.g., pass through a drying media) or contain a minimal amount of water or no water at all. Water can be removed from the reactant gases with any suitable method known in the art (e.g., condensation, liquid/gas separation, etc.).

In a particular aspect of the invention, the gaseous feed contains 1 wt. % or less, or 0.0001 wt. % to 1 wt. % of combined other reactant gas. In the reactant mixture, a molar ratio of methane to S(g) can range from 1:2 to 20:1 and any range therein (e.g., 1:1.9, 1:1.8, 1:1.7, 1:1.6, 1:1.5, 1:1.4, 1:1.4, 1:1.3, 1:1.2, 1:1.1, 1:1, 1.1:1, 1.2:1, 1.3:1, 1.4:1, 1.5:1, 1.6:1, 1.7:1, 1.8:1, 1.9:1, 2.0:1, 2.1:1, 2.2:1, 2.3:1, 2.4:1, 2.5:1, 2.6:1, 2.7:1, 2.8:1, 2.9:1, 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, 16:1, 16.1:1, 16.2:1, 16.3:1, 16.4:1, 16.5:1, 16.6:1, 16.7:1, 16.8:1, 16.9:1, 17:1, 17.1:1, 17.2:1, 17.3:1, 17.4:1, 17.5:1, 17.6:1, 17.7:1, 17.8:1, 17.9:1, 18.0:1, 18.1:1, 18.2:1, 18.3:1, 18.4:1, 18.5:1, 18.6:1, 18.7:1, 18.8:1, 18.9:1, 19.0:1, 19.1:1, 19.2:1, 19.3:1, 19.4:1, 19.5:1, 19.6:1, 19.7:1, 19.8:1, or 19.9:1). Preferably, the molar ratio of methane to S(g) is about 15:2 (7.5:1). Other ratios are also contemplated in the context of the present invention, however, the alkane (e.g., methane) is generally used in excess.

### C. Reaction Products

The products made from the reduction of methane with sulfur in the gas phase can be varied by adjusting the molar ratio of methane to S(g), the reaction conditions, or both. Without being limited by theory, a number of C₂+ hydrocarbons (e.g., ethane, ethylene, propane, propylene, butane, butylene, isobutene, pentane, pentene, etc.) can be produced in the processes of the current invention. In one aspect, the major products produced from the reaction of methane and S(g) can be ethylene (C₂H₄), hydrogen sulfide (H₂S), and hydrogen. Other carbon-based compounds can also be produced. For example, ethane (C₂H₆) and butadiene as shown in equation (2), can be present in the reaction product stream in an amount of 70 wt. % or less. Carbon disulfide and methanthiol can also be formed in amounts of less than 10 wt. % or less. In some aspects of the invention, the distribution of products in the product stream can be controlled by adjusting the ratio of methane to sulfur between 1:2 and 20:1, preferably around 5:1 to 10:1, or 6:1 to 9:1, or 7:1 to 8:1, more preferably about 7.5:1, and the temperature of the reaction.

8CH₄+4S₂→C₂H₄+8H₂S+C₂H₆+C₄H₆ (2)

### D. Oxidative Coupling of Methane Process

The reaction processing conditions using the catalysts of the current invention can be varied to achieve a desired result (e.g., ethylene product). In a preferred aspect, the process can include contacting a feed stream of alkane(s) and elemental sulfur with any of the catalysts described throughout the specification under established optimum OCM conditions (e.g., methane to sulfur ratio of 5:1 to 10:1 or preferably about 7.5:1, and reaction temperature of 750 to 950° C.) to afford a methane conversion of greater than 40% and an ethylene selectivity greater than 60%. In one aspect, the methane conversion is greater than about 40% and preferably greater than about 50%. In another aspect, the ethylene selectivity is greater than about 50% and preferably greater than about 70%.

In one aspect of the invention, the catalyst of the present invention can be used in continuous flow reactors to produce C₂+ hydrocarbons from methane (e.g., natural gas). Non-limiting examples of the configuration of the catalytic material in a continuous flow reactor are provided below and throughout this specification. The continuous flow reactor can be a fixed bed reactor, a stacked bed reactor, a fluidized bed reactor, or an ebullating bed reactor. In a preferred aspect of the invention, the reactor is a fixed bed reactor. The catalytic material can be arranged in the continuous flow reactor in layers (e.g., catalytic beds) or mixed with the reactant stream (e.g., ebullating bed).

In certain embodiments, a volume of catalyst in the contacting zone of a reactor is in a range from about 30 vol %, about 70 vol %, or about 60 vol % of a total volume of reactant in the contacting zone. Processing conditions in the reactor may include, but are not limited to, temperature, pressure, soft oxidant source flow (e.g., sulfur gas flow), hydrocarbon gas flow (e.g., methane or natural gas), ratio of reactants, or combinations thereof. Process conditions can be controlled to produce C₂ hydrocarbons with specific properties (e.g., percent ethylene, percent ethane, etc.). The average temperature in the reactor sufficient to produce a product stream includes a reaction temperature of at least 450° C. or 600° C. to 1100° C., preferably 750° C. to 950° C. and all values and ranges there between (e.g., 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, or 949° C.). Pressure in the reactor sufficient to produce a product stream can include a reaction pressure of 0.5 to 100 bar or 1 to 100, preferably between 5 and 25 bar and all values and ranges there between (e.g., 6, 7, 8, 9, 10, 11, 12, 13, 14, 15, 16, 17, 18, 19, 20, 21, 22, 23, or 24 bar). The gas hourly space velocity (GHSV) of the reactant feed can range from 500 h^{31 1} to 100,000 h⁻¹ or 1,000 to 50,000 h^{31 1} and all values and ranges there between (e.g., 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 3,0006, 37,000, 38,000, 39,000, 40,000, 41,000, 42,000, 43,000, 44,000, 45,000, 46,000, 47,000, 48,000, or 49,000 h^{31 1}). Preferably, the GHSV is between 3500 and 10,000 h^{31 1}, or 6000 h⁻¹ to 8500 h^{31 1}. In some embodiments, the GHSV is as high as can be obtained under the reaction conditions. Severity of the process conditions may be manipulated by changing the hydrocarbon source, the sulfur source, the reactant gas ratio, pressure, flow rates, the temperature of the process, the catalyst type, and/or catalyst to feed ratio.

Referring to FIG. 2, a schematic of system 10 for the production of olefins (e.g., ethylene) is depicted. System 10 may include a continuous flow reactor 12 and a catalytic material 14. In a preferred embodiment, catalytic material 14 is the catalyst of the present invention. A reactant stream that includes methane can enter the continuous flow reactor 12 via the feed inlet 16. A sulfur containing gas (soft oxidant) is provided via soft oxidant source inlet 18. In some aspects of the invention, the alkane(s) and the sulfur containing gas are fed to the reactor via one inlet (not shown). The reactants can be provided to the continuous flow reactor 12 such that the reactants mix in the reactor to form a reactant mixture prior to contacting the catalytic material 14. In some embodiments, the catalytic material and the reactant feed is heated to the approximately the same temperature. In some instances, the catalytic material 14 may be layered in the continuous flow reactor 12. Contact of the reactant mixture with the catalytic material 14 produces a product stream, for example, C₂+ hydrocarbons and generates heat (i.e., an exotherm or rise in temperature is observed). After contacting the catalyst, the reaction conditions are maintained downstream of the catalytic material at temperatures sufficient to promote continuation of the process. The product stream can exit continuous flow reactor 12 via product outlet 20.

In an exemplary embodiment, a method of producing an olefin from methane and elemental sulfur can include loading a catalyst of the present invention into a reactor (i.e., quartz reactor). The catalytic bed can be plugged with silicon carbide particulate to improve thermal uniformity along the bed. The silicon carbide can also be sandwich between two quartz wool plugs to keep the overall system fixed during operation. The reactor can then be place into a furnace and connected to a gas system and heated to a specific temperature and pressure (e.g., GHSV (N₂)=5000 h⁻¹, 850° C., 5 bar). At these conditions, the gas system can then be switched to a reactive mixture containing a methane, sulfur gas, and an inert gas to balance the space velocity. In some instances, the gaseous sulfur can be generated by heating elemental sulfur to 300° C. in a sealed reactor and passing the reactive gas (methane/nitrogen) through it. After the reaction, the product gas stream can be collected, analyzed, and/or subjected to further processing.

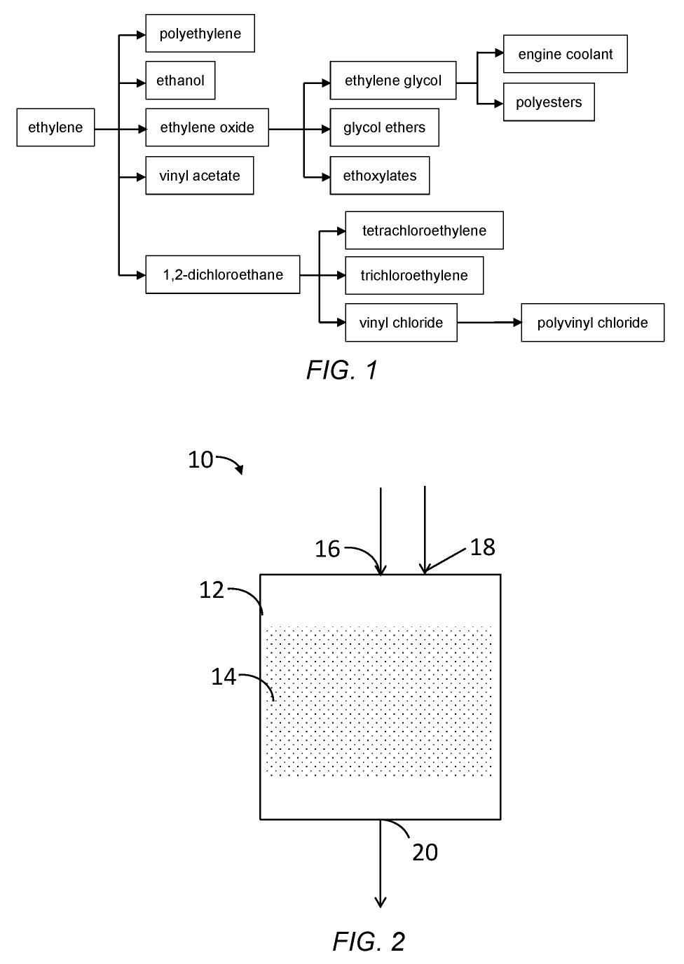

The resulting C₂+ hydrocarbons including olefins produced from the systems of the invention can be separated using gas/liquid separation techniques, for example, distillation, absorption, membrane technology to produce a gaseous stream that can include C₂+ hydrocarbons products (i.e., ethylene and ethane) and a H₂S stream. Other non-limiting methods used to separate H₂S from hydrocarbon gases can include the reaction with iron oxide, hydrodesulfurization, filtration through activated carbon, and plasma treatment. The separated or mixture of products can be used in additional downstream reaction schemes to create additional products or for energy production. Examples of other products include chemical products formed from ethylene such as the polyethylene, ethanol, ethylene oxide, vinyl acetate, 1,2-dichloroethane, etc., as shown in FIG. 1. H₂S can be further used for the production of thioorganic compounds (e.g., methanthiol, ethanthiol, thioglycolic acid, etc.), alkali metal sulfides (e.g., sodium hydrosulfide, sodium sulfide, etc.), metal sulfides, or for use in analytics, heavy water separation, or biologics. The method can further include isolating and/or storing the produced gaseous mixture or the separated products. H₂S and CS₂ can be burned to provide heat to the main reactor. Thus, the overall method provides a sustainable process for the production of olefins.

### EXAMPLES

The present invention will be described in detail by way of specific examples. The following examples are offered for illustrative purposes only, and are not intended to limit the invention in any manner. Those of skill in the art will readily recognize a variety of noncritical parameters, which can be changed or modified to yield essentially the same results.

### Example 1

### Preparation of a Manganese Sulfide

An aqueous manganese sulfate (8.45 g in 250 mL water) was added to a solution of sodium hydroxide (2M) under agitation until the formation of a precipitate. The precipitate was then recovered by filtration, vacuum dried at 120° C. for 2 hours, and then calcined at 1000° C. under inert atmosphere. The resultant MnO product was exposed to a sulfur source (i.e., H₂S) at 750° C. to be partially or entirely sulfidized.

### Example 2

### Preparation of a Praseodymium-Nickel Sulfide

An aqueous solution having an equimolar amount of praseodymium (10.87 g) and nickel nitrate (7.27 g) dissolved in water (100 mL) was prepared. Citric acid (14.32 g) was added into the praseodymium/nickel solution, followed by addition of ethylene glycol (4.65 g). The resulting mixture was heated to 85° C. until complete evaporation of the solvent to form a gel. The resulting gel was dried at 220° C. for 4 hours, and then calcined at 1000° C. The resulting mixed NiPr oxide having a perovskite-type structure was exposed to hydrogen sulfide at 850° C. for 6 hours to afford a mixed sulfide (e.g., NiPrSx).

### Example 3

### Preparation of a Titania-Iron Sulfide

In one example, commercial titania (about 80 m²/g surface area, 10 g) was first heated to 150° C. to remove absorbed/adsorbed water. After determination of the pore volume of the titania material, a solution, made by dissolving iron nitrate (5.12 g) in water (12 mL), was added to the solid and mixed. The resulting wet iron nitrate/titania solid was then dried and calcined at 900° C. under air for 3 hours and then transformed entirely or partially to FeS/titania under exposure to hydrogen sulfide.

### Example 4

### Prophetic Example for Sulfurization of CaGeO₃

In one sulfurization example, CaGeO₃ (2 g) will be placed into a dense alumina tube (OD: 25 mm, ID: 20 mm). At each extremity of the catalyst bed, a quartz wool pug will be placed to keep the material in a fixed position. The reactor will be connected to the sulfidation unit by swagelok type connection. The material will be heated to 800° C. under a nitrogen stream (150 sccm). When a set point is reached, H₂S (20%) will be added to the nitrogen gas mixture. The sulfurization process will then held for 46 min to afford CaGeO₂S analyzed by ex-situ XRD and online analysis using a mass spectrometer. Powder X-ray diffraction (XRD) patterns can be recorded on an PANalytical Empyrean X-ray diffractometer (PANalytical B.V., The Netherlands) using a nickel-filtered CuKα X-ray source, a convergence mirror and a PIXcel1d detector. The scanning rate will be 0.01° over the range between 5° and 80°2 θ. Online analysis was carried out using mass spectrometer (residual gas analysis) composed of a 100 amu source and quadrupole type detector and an analysis time of 50 ms per selected mass (H₂S was analyzed on mass 34, H₂O on mass 17, nitrogen on mass 28).

### Example 5

### Prophetic Example for Catalyst Testing

In a typical catalytic test, catalyst (100 mg, e.g., ZnMnO₂S₂) will be loaded in a quartz reactor (OD: 9 mm, ID 5 mm). The catalytic bed will then plugged with silicon carbide particulate to improve thermal uniformity along the bed. This silicon carbide will be sandwiched between two quartz wool plugs to keep the overall system fix during operation. The reactor will be placed into a furnace, and then connected to a gas system. The catalyst will be heated to 850° C. under nitrogen flow (GHSV=5000 h⁻¹) at 5 bar. When the desired temperature is reached the gas system will be switched to a reactive mixture containing a methane, sulfur and an inert gas to balance the space velocity with about a 7.5:1 to about 8:1 ratio between methane and sulfur. The gaseous sulfur will be generated by heating the elemental sulfur to 300° C. in a sealed container and passing the reactive gas (methane/nitrogen) through it. After the reaction, the unreacted sulfur can be trapped into a condenser and the gas effluent can be analyzed by a micro-gas chromatographer containing four modules to identify reactants and products.

1. A method of producing an olefin from methane and elemental sulfur, the method comprising:
(a) obtaining a reaction mixture comprising methane and elemental sulfur gas; and
(b) contacting the reaction mixture with a catalyst under reaction conditions sufficient to produce a product stream comprising an olefin, wherein the catalyst is a metal, a mixed metal oxide, mixed metal sulfide, a metal oxysulfide, mixed metal oxysulfide, or any mixture thereof.

2. The method of claim 1, wherein the olefin comprises C₂+ hydrocarbons, preferably ethylene.

3. The method of claim 1, wherein the product stream further comprises hydrogen sulfide.

4. The method of claim 1, wherein the reaction mixture comprises a methane to elemental sulfur molar ratio of 1:2 to 20:1.

5. The method of claim 1, wherein the conditions sufficient to produce a product stream in step (b) comprise a reaction temperature of at least 450° C.

6. The method of claim 1, wherein the conditions sufficient to produce a product stream comprise a reaction pressure of 0.05 to 10.0 MPa or 0.1 to 10.0 MPa, a gas hourly space velocity (GHSV) of 500 to 100,000 or both.

7. The method of claim 1, wherein the metal, the mixed metal oxide, the mixed metal sulfide, the metal oxysulfide, mixed metal oxysulfide, or the metal sulfide comprises:
an alkaline earth metal, preferably magnesium (Mg), calcium (Ca), strontium (Sr), barium (Ba), or any combination thereof;
a transition metal, preferably yttrium (Y), zirconium (Zr), vanadium (V), tantalum (Ta), tungsten (W), manganese (Mn), rhenium (Rh), iron (Fe), cobalt (Co), iridium (Ir), nickel (Ni), copper (Cu), zinc (Zn), or any combination thereof;
a post-transition metal, preferably aluminum (Al), gallium (Ga), indium (In), silicon (Si), germanium (Ge), tin (Sn), antimony (Sb), bismuth (Bi), or any combination thereof;
a lanthanide, preferably, lanthanum (La), cerium (Ce), praseodymium (Pr), neodymium (Nd), samarium (Sm), europium (Eu), gadolinium (Gd), or any combination thereof; or
any combination thereof.

8. The method of claim 1, wherein the catalyst does not include platinum sulfide, palladium sulfide molybdenum sulfide, titanium sulfide, ruthenium sulfide, tantalum sulfide, or combinations thereof.

9. The method of claim 1, wherein the catalyst does not include MgO, ZrO₂, TiO₂, CeO₂, Sm₂O₃, ZnO, W₂O₃, Cr₂O₃, La₂O₃ and Fe₃O₄.

10. The method of claim 1, wherein the catalyst comprises a spinel-, a halite-, a rutile-, or a perovskite-type crystal structure, or any combination thereof.

11. The method of claim 10, wherein the catalyst is an ordered mixture of one or more of the spinel-, halite-, rutile-, fluorite- or perovskite-type crystal structure.

12. The method of claim 10, wherein the catalyst has a spinel-type structure with a general formula of A²⁺B₂³⁺O_{4-y}²⁻S_{y}²⁻ where 0≤y≤4, or B₂O_{3-y}²⁻S_{y}²⁻ where 0≤y≤3, or A²⁺B′ₓ⁺³B₍₂₋ₓ₎³⁺O_{4-y}²⁻S_{y}²⁻ where 0≤x≤2 and 0≤y≤4 and A, B, and B′ are each independently an alkaline earth metal, a transition metal, a post transition metal or a lanthanide metal.

13. The method of claim 10, wherein the catalyst has a halite-type structure with a general formula A₁₋ₓBₓO_{1-y}S_{y}, where 0≤x≤1 and 0≤y≤1, and where A and B are each independently an alkaline earth metal, a transition metal, a post transition metal, or a lanthanide metal.

14. The method of claim 10, wherein the catalyst comprises a rutile-type structure with a general formula of A₁₋ₓBₓO_{2-y}S_{y}, where 0≤x≤1 and 0≤y≤2, and A and B are each independently an alkaline earth metal, a transition metal, a post transition metal, or a lanthanide metal.

15. The method of claim 10, wherein the catalyst comprises a perovskite-type structure with a general formula ABO_{3-y}²⁻S_{y}²⁻ where 0≤y≤3, and A and B are each independently an alkaline earth metal, a transition metal, a post transition metal, or a lanthanide metal, preferably CaGeO_{3-y}S_{y}, LaNbO_{3-y}S_{y}, PrNiO_{3-y}S_{y}, or NdGaO_{3-y}S_{y}, where 0≤y≤3, or a perovskite-type structure with a general formula A²⁺(B′ₓB₍₁₋ₓ₎)⁴⁺O_{3-y}²⁻S_{y}², wherein A, B can each independently be one or more of an alkaline earth metal, a transition metal, a post-transition metal or a lanthanide metal, 0.1≤x≤0.9, 0≤y≤3, and B′ is an alkaline earth metal, a transition metal, a post-transition metal or a lanthanide metal.

16. The method of claim 10, wherein the catalyst comprises a fluorite-type structure with a general formula AO₂₋ₓSₓ, ABO_{3.5-y}S_{y}, or A₂O_{3-z}S_{z}, where 0≤x≤2, 0≤y≤3.5, 0≤z≤3, and A and B are each independently an alkaline earth metal, a transition metal, a post transition metal, or a lanthanide metal.

17. The method of claim 9, wherein A and B are each individually an alkaline earth metal, a transition metal, a post-transition metal, or a lanthanide; wherein
A is a 2+ charged cation, preferably calcium (Ca), strontium (Sr), europium (Eu), indium (In), gallium (Ga), zinc (Zn), nickel (Ni), cobalt (Co), or copper (Cu), and
B, B₂, B′, or a combination thereof are a 3+ to 6+ charged cation that can change oxidation state to accommodate oxygen and/or sulfur, preferably manganese (Mn), iron (Fe), germanium (Ge), cerium (Ce), or bismuth (Bi).

18. The method of claim 1, wherein the catalyst is a bulk metal catalyst or a supported catalyst.

19. The method of claim 18, wherein the catalyst is a supported catalyst and the support comprises alumina, silica, titania, zirconia, magnesia, lime, silicon carbide, or combinations thereof, and, optionally, the support is macroporous, mesoporous, microporous, or any combination thereof.

20. A system for producing olefins from alkanes and elemental sulfur, the system comprising:
an inlet for a feed comprising a gaseous alkane(s) and elemental sulfur gas or a first inlet for a feed comprising a gaseous alkane(s) and a second inlet for a feed comprising elemental sulfur gas;
a reactor comprising a reaction zone that is configured to be in fluid communication with the inlet or inlets, wherein the reaction zone comprises gaseous alkane(s), elemental sulfur gas, and a catalyst capable of catalyzing the reaction between the alkane(s) and the sulfur gas to produce a product stream comprising a gaseous olefin(s), wherein the catalyst is a metal, a mixed metal oxide, mixed metal sulfide, a metal oxysulfide, a mixed metal oxysulfide, or any combination thereof; and
an outlet configured to be in fluid communication with the reaction zone to remove the product stream from the reactor.
